(19) United States
(12) Patent Application Publication
(10) Pub. No.: US 2002/0198560 A1
(43) Pub. Date: Dec. 26, 2002

(54) Flush tool and method for flushing air from catheters

(76) Inventors: William J. Boyle, Fallbrook, CA (US); Andy E. Denison, Temecula, CA (US); Benjamin C. Huter, Murrieta, CA (US); Scott J. Huter, Temecula, CA (US); Charles R. Peterson, Murrieta, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 09/894,161

(22) Filed: Jun. 26, 2001

(51) Int. Cl. A61M 29/00

(52) U.S. Cl. 606/194

(57) ABSTRACT

A flush tool provides a method of evacuating air from an intraluminal catheter. The tool has two valves which independently seal the tool to the catheter. In order to receive pressurized fluids the tool includes a flush port. When the tool is placed over the auxiliary lumen port of a rapid exchange catheter, it is capable of flushing air from the auxiliary lumen.

### BACKGROUND OF THE INVENTION

The present invention relates to a tool and device for flushing air from catheters. To better understand the need and uses of such a device, including the present invention, an understanding of catheters and their uses is required. Thus, the following paragraphs provide a brief discussion of medical catheters.

Catheters have long been used in intraluminal procedures for various medical needs. They generally are made from elongated tubes which may be placed within various body lumens. A common use for catheters is the treatment of vascular diseases. In such treatment, a catheter is inserted into a body vessel such as an artery. The catheter is then advanced through the artery to the site of the disease where treatment is performed.

A wide variety of treatments are currently available using different devices and mechanisms delivered with or within such catheters. For example, a stenosis of an artery may be treated by angioplasty. Stenosis of an artery is the narrowing of the body lumen due to vascular disease such as the build up of plaque. Angioplasty is a method of expanding the body lumen and compressing the plaque. This may be accomplished by expanding a balloon mounted on the catheter at the site of the narrowing. The catheter is inserted through the body lumen until the angioplasty balloon is at the site of the narrowing. The balloon is then expanded which compresses the plaque and expands the body vessel.

The above-described angioplasty procedure is just one example of the many uses of catheters which are currently employed. Other examples include the placement of grafts and stents, the capture and removal of emboli (blood clots), radiation treatments of vessels, etc. All such intraluminal procedures require the insertion and advancement of catheters through body vessels. Such procedures have become quite prevalent in modern medical practice.

Certain medical terms regarding the orientation of medical devices are useful for a complete understanding of the devices. The term “distal” typically refers to a direction away from the operator of the device. Thus, the distal end of the catheter is inserted into the body and advanced distally through the vascular system. The term “proximal” typically refers to a direction towards the operator of the device. Thus, the proximal end of the catheter remains outside of the body and the catheter is withdrawn proximally to be removed from the body. These terms will be used herein for consistency.

Intraluminal procedures typically employ the use of guide wires. Guide wires are typically thin, highly flexible wires which can be advanced through the body lumen prior to the introduction of the catheters. A physician can then deliver the catheter to the appropriate location by advancing the catheter over the guide wire.

A typical intraluminal procedure might proceed as follows: After accessing an artery by puncturing or incising the skin, the physician secures an access sheath through the opening. The physician then inserts a guide wire into the artery and advances it through the lumen to the site of a stenosis and beyond. The proximal end of the guide wire is then threaded into a port at the distal end of an angioplasty balloon catheter. This port accesses an internal lumen of the catheter which allows the catheter to be advanced distally over the guide wire. While the guide wire remains in place within the body the catheter may be advanced to the site of the stenosis. Once treatment is completed, the physician removes the catheter from the body while the guide wire remains in place. If necessary, other catheters may be advanced over the guide wire and withdrawn without removing the guide wire from the body. After all necessary treatment is completed the physician withdraws the guide wire. Thus a guide wire is often the first implement inserted into the vascular system and the last withdrawn.

Intraluminal procedures are often quite complex requiring the advancement and withdrawal of multiple catheters over a single guide wire. Using a conventional catheter in such circumstances consumes a lot of time and effort by the physician. In this context a conventional catheter is one that has a guide wire lumen extending from the distal end of the catheter to the proximal end of the catheter (called an “over-the-wire” catheter). In order to advance and withdraw such a catheter the entire catheter must be handled and supported. When withdrawing the catheter the proximal end must be supported until the distal end of the catheter is pulled away from the proximal end of the guide wire. Since these devices may be quite long, the procedure may require multiple operators to handle the catheters. This can be awkward, and since medical procedures require strict adherence to sterile procedures, missteps can be costly in terms of time and wasted devices.

A recent solution to such complications is the use of catheters which have been modified for rapid exchange. There are currently many such devices known to those of skill in the art. For example, the devices described and claimed in U.S. Pat. No. 4,748,982 (Horzewski, et al.); U.S. Pat. No. 5,040,548 (Yock); U.S. Pat. No.5,061,273 (Yock); U.S. Pat. No. 5,180,368 (Garrison) and U.S. Pat. No. 5,993,460 (Beitelia, et al.) all describe catheters which are modified for rapid exchange.

Many embodiments of rapid exchange catheters contain a separate lumen for the guide wire. This lumen extends from the distal end of the catheter to an access port in the side of the catheter. This access port is typically located several inches proximal of the balloon or other treatment device. In this configuration, the guide wire only extends through the portion of the catheter between the access port and the distal end. Proximal of the access port, the guide wire resides alongside the catheter. This allows the rapid exchange catheter to be withdrawn without need to continuously support and handle the proximal end of the catheter.

Prior to inserting any catheter into the body the physician must purge the air from the catheter. Otherwise, air bubbles in the catheter may escape into the bloodstream and cause an embolism. This can be a life-threatening condition.

In some instances, a physician may purge the air from an over-the-wire catheter by connecting a flush tool to the proximal end of the catheter which accesses the catheter lumens. Such a flush tool typically consists of a flush port and some means to seal the system proximal to the flush port. Pressurized fluid (typically saline) is then introduced into the flush port. This may be as simple as inserting a syringe over the flush port and forcing in fluid by hand. The fluid forces air out of the system through the distal end of the catheter lumen. Once fluid exits the distal end of the catheter lumen, the system is purged of air, or “flushed.”

Due to its structure, the guide wire lumen of a rapid exchange catheter is not accessible from the proximal end of the catheter system. The distal end of the catheter system may contain delicate implements which prevent the connection of a flush tool to that end. Air is usually purged from the guide wire lumen of rapid exchange catheters by inserting a syringe into the distal end of the catheter. At times this procedure may be awkward and time consuming as well.

What has been needed, is a tool and method for improving the ease and time needed to flush the air from the guide wire lumen of a rapid exchange catheter. This invention satisfies this and other needs.

### SUMMARY OF THE INVENTION

This invention relates to a tool for flushing air from an intraluminal medical device. More specifically, this tool is capable of flushing air from an auxiliary lumen of a catheter. To accomplish this task, the tool includes a proximal sealing valve, a distal sealing valve and a flush port. This invention also relates to the method of using this tool and for flushing a catheter equipped with a side port and auxiliary lumen.

What has been needed in the art of intraluminal devices is a tool and method of quickly and efficiently flushing the guide wire lumen of a rapid exchange (“Rx”) catheter. The device and method described herein meet these needs. The present invention mounts onto a catheter over the side port access to the guide wire lumen. The present invention then creates fluid tight seals proximal to, and distal to, the side port access. Air may then be flushed from the guide wire lumen by introducing pressurized fluid into the flush port of the device. The claimed method includes the procedure for performing these functions.

In another aspect of the present invention, the flush tool includes a tube having a first lumen which can be described as extending proximally to distally. The flush tool has a proximal valve located near the proximal end of the tube. The flush tool also has a distal valve located near the distal end of the tube. The tool also includes a flush tube having a flush port. The flush tube has a flush lumen in fluid communication with the first lumen.

The proximal valve and distal valve are capable of releasably sealing the flush tool about an object (such as a catheter) extending through the first lumen. They may also create seals without anything extending through the first lumen. The valves preferably create fluid-tight (possibly hemostatic) seals regardless of whether an object extends through the valve or not.

The flush tool of the present invention may include a great variety of valve-types. One such valve which may be included on the present invention is actuated by pulling a valve actuator mechanism proximally or distally with respect to the sealing mechanism. The proximal valve may be oriented such that the valve is sealed when the valve actuator mechanism extends proximally and the valve is opened by pushing the actuator mechanism distally with respect to the sealing mechanism. Contrary-wise the distal valve may be oriented such that the valve is sealed when the valve actuator mechanism extends distally and the valve is opened by pushing the actuator mechanism proximally with respect to the sealing mechanism.

A variant of the valve described above also includes a spring. The spring may bias the valve into the sealed position such that opening the valve requires constant pressure. Without pressure, the spring forces the actuator mechanism away from the sealing mechanism thereby sealing the valve. A similar device may also be constructed in which the valve is biased into the open position.

Another type of valve may be actuated by twisting or rotating the actuating mechanism relative to the sealing mechanism. One such device is known in the art as a Touhy-Borst valve. One advantage of such a valve is that by regulating the amount of rotation on the actuator mechanism, the force applied by the sealing mechanism may be varied. Thus, a very tight seal may be used where required, or alternatively, a less tight seal may be used to avoid crushing components extending through the valve.

These and other valve-types may be used in different combinations with the present invention. For example, a pushing actuated valve can be attached to one end and a twisting actuated valve attached to the other. In another example, the pushing actuated valve and the twisting actuated valve can be combined into a single valve which can be operated either by pushing or twisting. These, and other examples permit a variety of actuating means to be employed on different embodiments of devices all within the scope of the present invention.

One feature of the present invention includes the use of tabs on the actuating mechanism of one or both valves on the flush tool. These tabs may allow the valves to be actuated together and in a single motion. For instance, tabs may be situated on each of a pair of pushing-type actuated valves such that by pinching the tabs together both valves would be opened. Upon releasing the tabs, the valves may then both seal if each valve includes a biasing spring. Alternatively, valves not having biasing springs could be resealed by forcing the tabs apart. Similarly, tabs may be located on each of a pair of twisting-type actuated valves to allow the valves to twist open or twist sealed by simply grasping both tabs.

The present invention is particularly useful in combination with an Rx-type catheter. These catheters are characterized by having a side port in relative proximity to the distal end of the catheter. This side port accesses an auxiliary lumen of the catheter which then extends to the catheter distal end. This auxiliary lumen is configured to glide over the guide wire and facilitates the rapid exchange of the catheter without removing the guide wire from the patient. The flush tool of this invention may be combined with such a catheter to flush the auxiliary (or guide wire) lumen.

The flush tool may be removably sealed to the Rx catheter about the side port. Thus, the catheter itself extends through the first lumen, the proximal valve and the distal valve of the flush tool. Sealing the respective valves over the catheter proximal to, and distal to, the side port maintains the flush port of the flush tool in fluid-tight communication with the side port of the catheter and the guide wire lumen. The guide wire lumen may then be flushed by introducing pressurized fluid into the flush port.

The process for flushing the guide wire lumen may include additional steps to ease the procedure. For instance, a peel-away introducer sheath may be included throughout the first lumen of the flush tool to aid the mounting of the flush tool over the catheter. The peel-away introducer sheath resides within the first lumen and isolates the protuberances caused by the valves and flush port. Thus the introducer sheath provides a smooth and uninterrupted inner lumen. Moreover, the process for flushing may include flushing air from the flush tool itself prior to flushing the auxiliary lumen.

The advantages of the present invention will become apparent from the following detailed description thereof when taken in conjunction with the accompanying exemplary drawings. Those of skill in the art will recognize that variations are possible to the described preferred embodiments while remaining within the scope of the present invention. Thus, the following description are not intended to limit the scope of the invention which is to be defined by the claims.

### BRIEF DESCRIPTION OF THE DRAWINGS

FIG. 1 is a side view of a flush tool conforming to the present invention.

FIG. 2 is an end view of the flush tool in FIG. 1.

FIG. 3 is an exploded cross-sectional side view of a flush tool conforming to the present invention.

FIG. 4 is an enlarged, exploded cross-sectional sideview of a valve on the distal end of a flush tool.

FIG. 5 is a side view of a flush tool conforming to the present invention and a peelable introducer sheath.

FIG. 6 is a side view of a catheter and a guide wire system.

FIG. 7 is a cross-sectional view taken along line 7-7 in FIG. 6.

FIG. 8 is a cross-sectional view taken along line 8-8 in FIG. 6.

FIG. 9 is a cross-sectional view taken along line 9-9 in FIG. 6.

FIG. 10 is a side view of a system for flushing an auxiliary lumen in accordance with the present invention.

### DETAILED DESCRIPTION OF THE PREFERRED EMBODIMENTS

The present invention is directed to a tool and method for flushing air from a catheter. In particular, the present invention is directed to a flush tool which can be mounted over a side port access in an auxiliary lumen in a catheter and the process of flushing that lumen.

While the invention is described in detail as applied to a rapid exchange catheter, those skilled in the art will appreciate that it can be used on any catheter having an auxiliary lumen extending between a side port and a distal port.

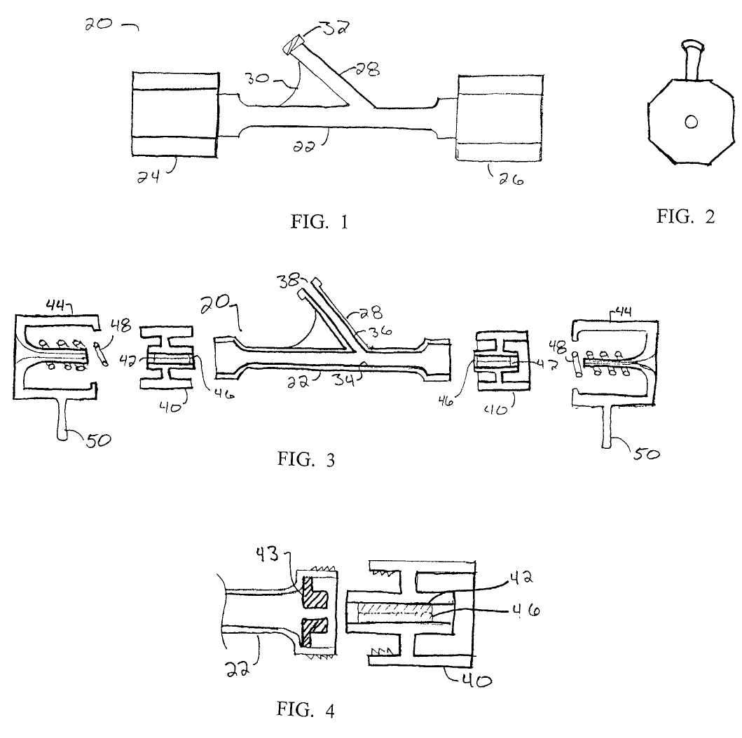

FIG. 1 depicts a side view of an embodiment of the present invention. A flush tool 20 is shown which is generally comprised of a main tube 22 which forms the body of the tool. A proximal valve 24 is located at the proximal end of the main tube. A distal valve 26 is located at the distal end of the main tube. Attached to the main tube between the proximal end and the distal end is a flush tube 28. The flush tube connects to the main tube at an angle. To provide structural support of the flush tube a flange 30 may connect the flush tube and main tube. The flush tube may also include a threaded end fitting 32 for connecting to a variety of devices.

FIG. 3 depicts an exploded cross-sectional side view of the present invention, and the inner workings of the flush tool 20. The main tube 22 houses a main lumen 34 (also described as the first lumen). While the proximal valve 24 and distal valve 26 are in the open position, the opening of the main lumen extends throughout the entirety of the flush tool. Likewise, the flush tube 28 houses a flush lumen 36. The flush lumen connects with the main lumen 34 so that fluid may flow freely between the two lumens. A flush port 38 located at the opposite end of the flush lumen allows introduction of fluid to the flush tool 20 from an external source.

The valves of the flush tool 20 may have a variety of configurations. In one embodiment of the flush tool, both the proximal valve 24 and distal valve 26 include a valve sealing member 40 and a valve actuating member 44. The valve sealing members each connect to the ends of the main tube 22 and house a seal 42. The connection between the sealing members and the main tube may include mating threaded ends. The seals are typically formed of a pliable material and have either a slit or pinhole extending through the material. The pliable nature of the seal keeps the pinhole closed forming a fluid-tight (possibly hemostatic) seal. The valve actuating member fits slidably over the valve sealing member. The valve actuating member is configured such that when it is slid toward the valve sealing member it permits external access to the main lumen 34 through the valve by forcing open the pinhole of the seal. When the valve actuating member slides away from the valve sealing member the pinhole closes resealing the valve.

The valves may also include a biasing spring 48. The biasing spring forces the valve into the sealed position (as depicted in FIG. 3). The biasing spring in such a configuration connects between the valve sealing member 40 and the valve actuating member 44. When pressure is not actively being applied to the actuating member to keep the valve in the open condition, the biasing spring will force the valve actuating member away from the valve sealing member and thereby seal the valve.

Another component which may be included on the flush tool 20 is a tab 50 connected to one or both of the valves. Preferably, a tab is included on both the proximal valve 24 and the distal valve 26. In this configuration (as depicted in FIG. 3) both valves may be simultaneously actuated into the open position by pinching the tabs together. This might be achieved between the thumb and forefinger for ease of use. In this configuration, both valves may also be simultaneously actuated into the closed position.

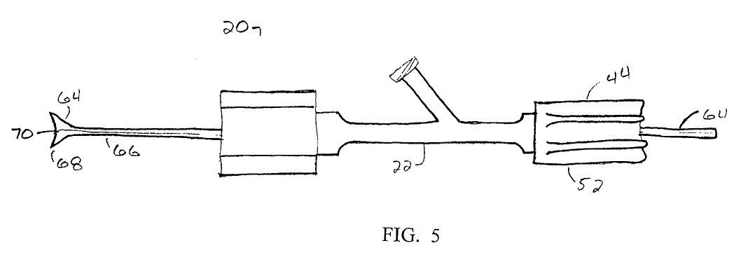

Another embodiment of the flush tool 20 may include a twisting-type actuated valve 52 on one or both ends. FIG. 5 depicts such a valve on the distal end of the main tube 22. One such valve is known to those of skill in the art as a Touhy-Borst valve. These valves are sealed by twisting the valve actuating member 44 which compresses the seal 42 into a closed position. This type valve may be particularly useful when a very tight seal is required.

Yet another embodiment of the present invention may include valves which are actuated both by pushing or twisting the valve actuating member 44. Such a valve is depicted in FIG. 4. Such a valve includes a first seal 42, and a second seal 43. The first seal has a pinhole 46 which is opened by pushing a portion of the valve actuating member through the pliable material of the seal. The second seal is disposed within the main tube 22. This seal closes when the valve actuating member is twisted. Twisting the valve actuating member causes the valve sealing member 40 to compress against the pliable material of the seal. Transferring the twisting motion into a compressive force is enabled by a threaded engagement between the valve sealing member and the main tube.

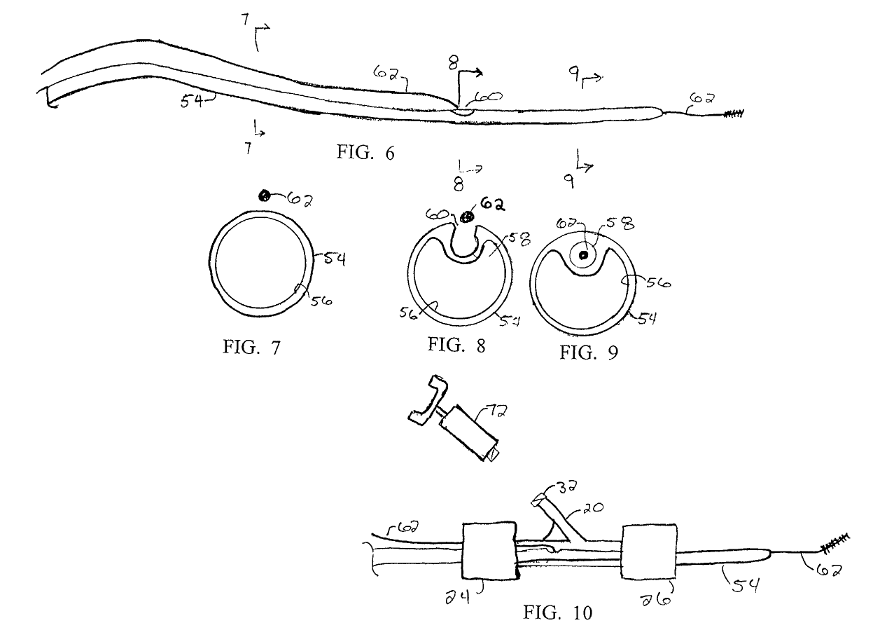

FIGS. 6-9 depict a simple form of a catheter 54 which may benefit from the present invention. The catheter includes a main catheter lumen 56 which extends throughout the length of the catheter. The catheter also includes an auxiliary lumen 58 which extends from the distal tip of the catheter to a side port 60 located in relative proximity to the distal tip. That is, while the entire catheter may be three feet or more in length, the side port is typically within several inches of the distal tip of the catheter.

As depicted in FIGS. 6-9 the auxiliary lumen 58 may be configured as a guide wire lumen. This configuration typifies the rapid exchange (“Rx”) catheter. As such, a guide wire 62 extends throughout the auxiliary lumen 58 while in use. Guide wires generally extend beyond the distal tip of the catheter 54. Extending proximally, the guide wire exits the side port 60 and thereafter resides next to the catheter. Those of skill in the art will recognize this as a simple example of an Rx catheter for descriptive purposes. Rx catheters are typically much more complex. Some examples of Rx catheters include angioplasty catheters, stent delivery catheters and embolic protection device catheters. Each of these catheters is more complex than that described herein. Typically between the catheter distal tip and the side port, these more complex catheters will include devices to perform the intended functions. Importantly, Rx catheters include a side access port, and an auxiliary lumen which extends from the side access port to the catheter distal tip as described herein. The present invention is intended for use with catheters having these common features.

Prior to use, catheters require flushing. That is, air must be evacuated from the entire catheter system. The main catheter lumen(s) is (are) typically flushed by introducing pressurized fluid into the proximal end of the catheter. Once the fluid exits the distal tip of the catheter, the air has been purged from that lumen. The fluid used in this process is typically saline or heparinized saline.

The method of the current invention includes using the flush tool 20 described herein to flush the auxiliary lumen 58 of the catheter 54. While both the proximal valve 24 and the distal valve 26 are in the open position, the catheter 54 may be inserted through the main lumen 34 until the main tube 22 is located over the side port 60 of the catheter. The main tube may be formed of a clear plastic material to facilitate visualization when centering the side port within the flush tool 20.

The flush tool 20 may also include a peel-away introducer sheath 64 to ease the introduction of the catheter 54 into the main lumen 34. Such a sheath may include a plastic tube 66 with a funnel shape 68 on one end and seam 70 splitting the entire device along its length. The introducer sheath initially extends throughout the main lumen providing a smooth uninterrupted interior lumen. A catheter may then be inserted into the funnel shape end of the introducer sheath and advanced through the introducer sheath and the flush tool. This prevents the catheter or guide wire from becoming snagged on the valves or flush lumen 36. Once the catheter is in place, the introducer sheath may be peeled away along the seam, removed and discarded.

Once the flush tool 20 is in place over the side port 60 on the catheter 54, air may be purged from the flush tool. By alternatively sealing the proximal valve 24 and the distal valve 26 while leaving the opposing valve open, pressurized fluid may be introduced into the flush port 38 to force air out of the distal end and then the proximal end of the flush tool respectively. Pressurized fluid may be introduced into the flush port by use of a syringe 72. The end of the syringe may be inserted over the flush lumen 36. By depressing the plunger on the syringe, the fluid within the syringe is pressurized and forced into the main lumen 34 of the flush tool 20. If a tight seal is required between the syringe and flush tool an appropriately configured syringe may be threaded onto the threaded end fitting 32 of the flush tube 28. Pressurized fluid may also be introduced by connecting a pressurized pouch of fluid (not shown) onto the flush port.

To flush the auxiliary lumen 58 with fluid, each of the proximal valve 24 and distal valve 26 are placed in the sealed condition about the catheter 54 with the side port 60 centered within the main lumen 34. Pressurized fluid is then introduced into the flush port 28. Since all other exits from the flush tool have fluid-tight seals, the fluid is forced into the side port and the auxiliary lumen 58. Once fluid exits the auxiliary lumen at the distal tip of the catheter 54, the lumen is flushed of air. The flush tool may then be removed form the catheter by unsealing the valves and withdrawing the catheter.

Another possible method of using the flush tool 20 to flush air from the auxiliary lumen 58 of a catheter 54 employs the creation of a vacuum force at the flush port 38. In this method the distal end of the catheter is immersed in fluid. With the flush tool tightly sealed about the side port 60, a vacuum force is created at the flush port. This may also be accomplished using a syringe 72. The vacuum force then draws fluid into the auxiliary lumen from the distal end of the catheter. When fluid is drawn into the flush tool from the side port, the auxiliary lumen has been flushed.

The method of the present invention is particularly useful when the auxiliary lumen 58 is occupied by devices which obstruct the free flow of fluid. One situation in which this condition exists is when the guide wire 62 has already been threaded through the auxiliary lumen. Furthermore, some configurations of Rx catheters require loading of devices into the guide wire lumen. One such device is an embolic protection device, which is connected near the distal tip of the guide wire. This device resides preloaded in the guide wire lumen of the delivery catheter. Of course, the methods described herein are also useful when the auxiliary lumen is unoccupied.

The present invention is also particularly useful in such instances because of its ability to create strong fluid-tight seals. Thus the higher pressures required to flush an occluded auxiliary lumen will not cause leaking from the flush tool 20. The twisting-type actuated valve 52 may be of ideal use in these conditions due to its ability to create an exceptionally tight seal.

The dimensions of the flush tool 20 may vary in accordance with the intraluminal device to be flushed. In particular, the diameter of the main lumen 34 should be sized to allow the distal end of the catheter (including the portion containing the functional devices) to be readily inserted and withdrawn without creating an excessively large space to flush. The length of the main tube can be varied to allow the seals 42 of the valves to engage the catheter at appropriate locations. Furthermore, other dimensions of the device may be varied without evading the scope of the present invention.

Intraluminal procedures can be quite complex. Providing an Rx catheter with a flush tool 20 of the present invention pre-mounted over the side port 60 may simplify the use of the present invention. Anything that simplifies the overall procedure will be seen as an advantage.

The flush tool and all its components may be formed from many well-known plastics. Injection moldings and similar procedures are proven techniques of manufacturing medical quality tools. Those of skill in the art will readily recognize that many materials and manufacturing methods may be utilized to form the present invention. Thus, the scope of the present invention is intended to be limited only by the following claims:

We claim:

1. A flush tool for medical catheters, comprising:
a first tube having a proximal end and a distal end;
a flush tube in fluid communication with the first tube;
a first valve attached to the proximal end of the first tube and in sealable fluid communication with the first tube; and
a second valve attached to the distal end of the first tube and in sealable fluid communication with the first tube.

2. The flush tool of claim 1, wherein,
the first tube is configured to encompass a portion of a catheter and the first valve and second valve are each configured to sealably engage the catheter.

3. The flush tool of claim 1, wherein the first valve comprises a sealing member attached to the proximal end of the first tube and an actuating member slidably engaged with the sealing member which opens the sealing member when compressed with the sealing member.

4. The flush tool of claim 3, wherein the second valve comprises a sealing member attached to the distal end of the first tube and an actuating member slidably engaged with the sealing member which opens the sealing member when compressed with the sealing member.

5. The flush tool of claim 3, wherein the first valve further comprises a spring which biases the actuating member away from the sealing member, thereby biasing the first valve into a sealed condition.

6. The flush tool of claim 4, wherein the second valve further comprises a spring which biases the actuating member away from the sealing member, thereby biasing the second valve into a sealed condition.

7. The flush tool of claim 4, wherein the first valve further comprises a first tab on the actuating member, and the second valve further comprises a second tab, whereby the first valve and the second valve may be simultaneously actuated by gripping both the first tab and the second tab.

8. The flush tool of claim 1, wherein the first valve comprises a sealing member attached to the proximal end of the first tube and an actuating member rotatably engaged with the sealing member and configured to seal the valve by rotating about the sealing member.

9. The flush tool of claim 1, wherein the second valve comprises a sealing member attached to the distal end of the first tube and an actuating member rotatably engaged with the sealing member and configured to seal the valve by rotating about the sealing member.

10. The flush tool of claim 1, wherein the first tube is formed of a transparent material, whereby items within the first tube may be visually inspected.

11. The flush tool of claim 1, wherein the flush tube is equipped with a means for attaching to a fluid source.

12. A system for flushing a catheter, comprising:
a catheter having an auxiliary lumen extending from a distal end of the catheter to a side port in the catheter;
a flush tool having a proximal end and a distal end mounted on the catheter over the side port;
a first fluid-tight seal located near the proximal end of the flush tool and sealed about the catheter;
a second fluid-tight seal located near the distal end of the flush tool and sealed about the catheter; and
a flush port attached to the flush tool;
whereby introducing pressurized fluid into the flush port will evacuate air from the auxiliary lumen of the catheter.

13. The system of claim 12, wherein the flush tool is removable from the catheter by unsealing the first fluid-tight seal and the second fluid-tight seal.

14. The system of claim 12, further comprising:
a guide wire extending through the auxiliary lumen.

15. The system of claim 12, further comprising:
an intraluminal device within the auxiliary lumen which impedes the flow of fluid therethrough.

16. The system of claim 12, further comprising:
a syringe connected to and in fluid communication with the flush port.

17. The system of claim 12, wherein the first fluid-tight seal and the second fluid-tight seal may be independently and repeatedly sealed and unsealed.

18. The system of claim 12, whereby introducing vacuum pressure on the flush port will draw fluid into the auxiliary lumen at the distal end of the catheter through to the side port.

19. A method of flushing air from the guide wire lumen of a rapid exchange catheter wherein the guide wire lumen extends from a side access in the catheter to the distal end of the catheter, comprising the steps of:
placing a flush tool having a flush port over the catheter;
sealing the flush tool to the catheter distally from the side access;
sealing the flush tool to the catheter proximally from the side access; and
introducing pressurized fluid into the flush port.

20. The method of claim 19, further comprising the steps of:
introducing pressurized fluid into the flush port prior to sealing the flush tool to the catheter to force air out of the flush tool.

21. The method of claim 19, wherein the rapid exchange catheter includes a guide wire disposed throughout the guide wire lumen.

22. The method of claim 19, further comprising the steps of:
disposing an introducer sheath within the flush tool prior to placing the flush tool over the catheter; and
removing the introducer sheath from the flush tool after placing the flush tool over the catheter.
